(19) United States
(12) Patent Application Publication
(10) Pub. No.: US 2017/0110781 A1
(43) Pub. Date: Apr. 20, 2017

(54) SENSOR AND METHOD FOR MANUFACTURING THE SENSOR

(71) Applicant: LG ELECTRONICS INC., Seoul (KR)

(72) Inventors: Myungwon LEE, Seoul (KR); Hyunchul KIM, Seoul (KR); Daehyun PARK, Seoul (KR); Sanggyu KIM, Seoul (KR); Seungyeon SONG, Seoul (KR); Hongyoon JUNG, Seoul (KR)

(73) Assignee: LG ELECTRONICS INC., Seoul (KR)

(21) Appl. No.: 15/285,253

(22) Filed: Oct. 4, 2016

(30) Foreign Application Priority Data
Oct. 20, 2015 (KR) 10-2015-0146131
Mar. 14, 2016 (KR) 10-2016-0030487

(51) Int. Cl. H01Q 1/22; G01N 33/03; G01N 27/04; H01Q 7/00; H02J 50/10

(52) U.S. Cl. CPC H01Q 1/22; H01Q 7/00; H02J 50/10; G01N 27/04; G01N 33/03

(57) ABSTRACT

A sensor according to the present disclosure may include a substrate, an antenna pattern formed to transmit and receive a wireless signal to and from an external device, a sensing unit configured to be driven when the wireless signal is received through the antenna pattern and to generate a signal when in contact with a sensing target material, and a circuit line electrically connected between the antenna pattern and the sensing unit, wherein the antenna pattern and the circuit line are formed of a same material and on a same layer. A fabrication method of a sensor according to the present disclosure may include printing a conductive layer having an antenna pattern, a sensing electrode and a circuit line on one surface of a substrate with a single layer, heat-drying the conductive layer, printing, on a single layer, a circuit insulating layer that covers part of the circuit line and an antenna insulating layer that covers part of the antenna pattern, curing the insulating layer, printing an antenna bridge on the antenna insulating layer, heat-drying the antenna bridge, and bonding a device electrically connected to the circuit line to the substrate.

### CROSS-REFERENCE TO RELATED APPLICATION

Pursuant to 35 U.S.C. §119(a), this application claims the benefit of earlier filing date and right of priority to Korean Application No. 10-2015-0146131, filed on Oct. 20, 2015 and Korean Application No. 10-2016-0030487, filed on Mar. 14, 2016, the contents of which is incorporated by reference herein in its entirety.

### FIELD OF THE DISCLOSURE

The present disclosure relates to a sensor configured to sense the salt content, sugar content of materials, the rancidity of oil or the like and a method of fabricating the same.

### BACKGROUND OF THE DISCLOSURE

In recent years, the number of adult patients such as hypertension, hyperlipidemia, diabetes and the like has surged due to changes in eating habits, thus becoming a social issue. However, in many cases, the disease is usually found in an advanced stage to a certain level, thereby increasing social costs (costs for medical care and costs for treatment). In addition, in order to prevent adult diseases, the public interest in the safety of foods has increased, and the interest in whether pesticide residues in vegetables or fruits are safe or not has also increased.

However, the individual examination of various detection information, such as the safety of food, costs a lot and is accompanied by inconvenience. Furthermore, the reality is that it is very inconvenient and difficult to comprehensively and consistently manage the safety of food.

In the related art, a terminal type of sensor has been used to determine the salt content or sugar content of foods or the modification of edible fat and oil or the like. The terminal type of sensor has an advantage capable of independently examining the salt content of food or the like, and providing the examination result to a user. Meanwhile, the terminal type of sensor has several disadvantages as follows.

First, the terminal type of sensor needs additional power or a battery for the operation, and cannot be operated in an environment in which it is difficult to supply power.

In addition, the unit price of the terminal type of sensor is relatively high, thus making it difficult to become popular with the general public.

Finally, the terminal type of sensor is inconvenient to carry.

Accordingly, the development of a sensor capable of solving the foregoing disadvantages of the terminal type of sensor is needed.

### SUMMARY OF THE DISCLOSURE

An object of the present disclosure is to provide a sensor having a structure that can be fabricated with a simplified process and a low cost. Furthermore, the present disclosure is to propose a method of fabricating a sensor with a simplified process and a low cost

Another object of the present disclosure is to present a sensor having a configuration capable of suppressing the oxidation or corrosion of a sensing electrode.

Still another object of the present disclosure is to propose a sensor that can be driven by wireless communication with an external device without having a battery. Furthermore, the present disclosure is to provide a sensor having enhanced portability.

Yet still another object of the present disclosure is to provide a sensor capable of detecting glucose contained urine.

Still yet another object of the present disclosure is to provide a sensor having a structure that is advantageous for mass production as well as capable of solving a process error problem.

In order to accomplish an object of the present disclosure, there is provided a sensor according to an embodiment of the present disclosure may include a substrate, an antenna pattern formed to transmit and receive a wireless signal to and from an external device, a sensing unit configured to be driven when the wireless signal is received through the antenna pattern and to generate a signal when in contact with a sensing target material, and a circuit line electrically connected between the antenna pattern and the sensing unit, wherein the antenna pattern and the circuit line are formed of a same material and on a same layer.

According to an embodiment, the sensing unit may be driven by generating direct-current power from the wireless signal received through the antenna pattern and transmit the signal to the external device.

According to an embodiment, the circuit line may include a power generation unit configured to generate direct-current power using the wireless signal received through the antenna pattern, a controller configured to be driven by the direct-current power to input a voltage to the sensing unit, a conversion unit configured to convert the signal generated from the sensing unit into a digital signal, and a communication unit configured to transmit the digital signal to the external device through the antenna pattern.

According to an embodiment, the sensing unit may be an sensing electrode configured to cause an impedance change when in contact with the sensing target material, and the antenna pattern, the circuit line and the sensing electrode may be formed on a surface of the substrate as a single conductive layer.

According to an embodiment, the antenna pattern, the circuit line and the sensing electrode may be formed of the same material.

According to an embodiment, the sensor may include a circuit insulating layer provided with a window to expose the sensing electrode, where the circuit insulating layer is disposed to cover at least part of the circuit line.

According to an embodiment, the sensing electrode may include a first end portion and a second end portion in a lengthwise direction, the circuit insulating layer may be formed to cover the first end portion and the second end portion, and the window may be formed to expose a region between the first end portion and the second end portion.

According to an embodiment, a length of the sensing electrode is more than 400 μm, and an exposure length of the sensing electrode exposed through the window may be 300-2,000 μm.

According to an embodiment, the circuit insulating layer may have surface energy larger than that of the substrate.

According to an embodiment, the single conductive layer may include solid particles formed of at least one of silver (Ag), copper (Cu) or aluminium (Al) to have a spherical shape or a flake shape, and at least one organic substance selected from a group consisting of polyethylene oxide (PEO) series, oleic acid series, acrylate series, acetate series and epoxy series.

According to an embodiment, the single conductive layer may have a pore.

According to an embodiment, the substrate may include a plastic layer having flexibility, and a silica layer formed between the plastic layer and the conductive layer.

According to an embodiment, the sensing unit may be a field-effect transistor including a gate electrode, a source electrode, and a drain electrode, and the field-effect transistor may include a channel layer located between the source and the drain electrode, an insulating layer located on the channel layer, and a sensing layer located on the insulating layer, wherein the sensing layer is separated from the gate electrode by a predetermined distance.

According to an embodiment, the field-effect transistor may be configured to cause a change in current value flowing along the channel layer when in contact with the sensing target material.

According to an embodiment, the sensing layer may be formed of a mixture of a predetermined enzyme and a predetermined high molecular weight compound.

According to an embodiment, the predetermined enzyme may include at least one of glucose oxidase or glucose dehydrogenase.

According to an embodiment, the sensing layer may include a self-assembled monolayer and a predetermined enzyme.

According to an embodiment, the antenna pattern may be extended in a two-dimensional spiral shape having a line width of 500-1,500 μm, and a distance between lines that form the two-dimensional spiral shape may be 300-700 μm.

Furthermore, there is provided a method of fabricating a sensor, and the method may include printing, on a single layer, a conductive layer having an antenna pattern, a sensing electrode and a circuit line on one surface of a substrate, heat-drying the conductive layer, printing a circuit insulating layer that covers part of the circuit line and an antenna insulating layer that covers part of the antenna pattern with a single layer, curing the insulating layer, printing an antenna bridge on the antenna insulating layer, heat-drying the antenna bridge, and bonding a device electrically connected to the circuit line to the substrate.

### BRIEF DESCRIPTION OF THE DRAWINGS

The accompanying drawings, which are included to provide a further understanding of the disclosure and are incorporated in and constitute a part of this specification, illustrate embodiments of the disclosure and together with the description serve to explain the principles of the disclosure.

In the drawings:

FIG. 1 is a plan view illustrating a sensor according to the present disclosure;

FIG. 2 is an exploded perspective view illustrating a sensor according to the present disclosure;

FIG. 3 is a cross-sectional view taken along line A-A in FIG. 1;

FIG. 4 is a conceptual view illustrating an interrelated operation between an external device and a sensor;

FIG. 5 is a flow chart illustrating a method of measuring the state of a measurement target material using a sensor;

FIG. 6 is a conceptual view illustrating the structure of a circuit line, a sensing electrode and a circuit insulating layer that are advantageous for mass production;

FIGS. 7 and 8 are graphs experimentally illustrating a relationship between the structure and resolution of a sensing electrode;

FIG. 9 is a flow chart illustrating a method of fabricating a sensor;

FIGS. 10A through 10D are conceptual views partially illustrating a fabrication process of a sensor according to the fabrication method of FIG. 9;

FIGS. 11A and 11B are conceptual views illustrating a field-effect transistor according to the present disclosure;

FIG. 12 is a circuit diagram illustrating a sensor including a field-effect transistor according to the present disclosure;

FIG. 13 is an exploded perspective view illustrating a sensor illustrated in FIG. 12; and

FIG. 14 is a conceptual view illustrating an embodiment of selecting a contact target material to be detected using an external device.

### DETAILED DESCRIPTION OF THE DISCLOSURE

Hereinafter, a sensor and a fabrication method thereof according to the present disclosure will be described in detail with reference to the accompanying drawings. Even in different embodiments according to the present disclosure, the same or similar reference numerals are designated to the same or similar configurations, and the description thereof will be substituted by the earlier description. Unless clearly used otherwise, expressions in the singular number used in the present disclosure may include a plural meaning.

A sensor according to the present disclosure may include a substrate, an antenna pattern, a sensing unit, and a circuit line.

Here, a sensor according to the present disclosure may be driven in different ways according to the type of the sensing unit.

Specifically, the sensing unit may be either one of a sensing electrode and a field-effect transistor. Hereinafter, a case where the sensing unit is a sensing electrode and a case where the sensing unit is a field-effect transistor will be separately described.

First, a case where the sensing unit is the sensing electrode will be described.

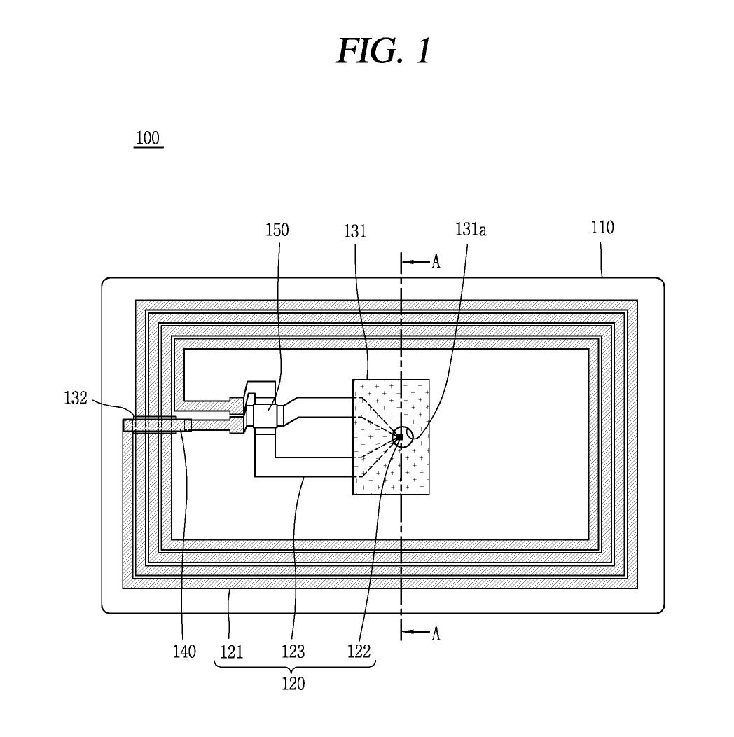

FIG. 1 is a plan view illustrating a sensor 100 according to the present disclosure.

The sensor 100 may include a substrate 110, a conductive layer 120, a circuit insulating layer 131, an antenna insulating layer 132, an antenna bridge 140, and a device 150. The constituent elements will be sequentially described below.

The casing 110 has a flat plate shape as a whole. The substrate 110 is configured to support the conductive layer 120, circuit insulating layer 131, antenna insulating layer 132, antenna bridge 140 and device 150. The conductive layer 120, circuit insulating layer 131, antenna insulating layer 132, antenna bridge 140 and device 150 may be formed or mounted on the substrate 110 by various processes.

The substrate 110 has flexibility. The sensor 100 of the present disclosure may be formed with a very small thickness, and thus easily damaged by an external force unless the substrate 110 has flexibility. However, when the substrate 110 has flexibility, the sensor 100 may have high reliability even at a repetitive mechanical deformation.

The substrate 110 is formed of plastic (e.g., polymer compound or synthetic resin) having flexibility. The plastic may include at least one selected from a group consisting of polyethylene terephthalate (PET), polyimide (PI), polystyrene (PS) and polyethylene naphthalate (PEN).

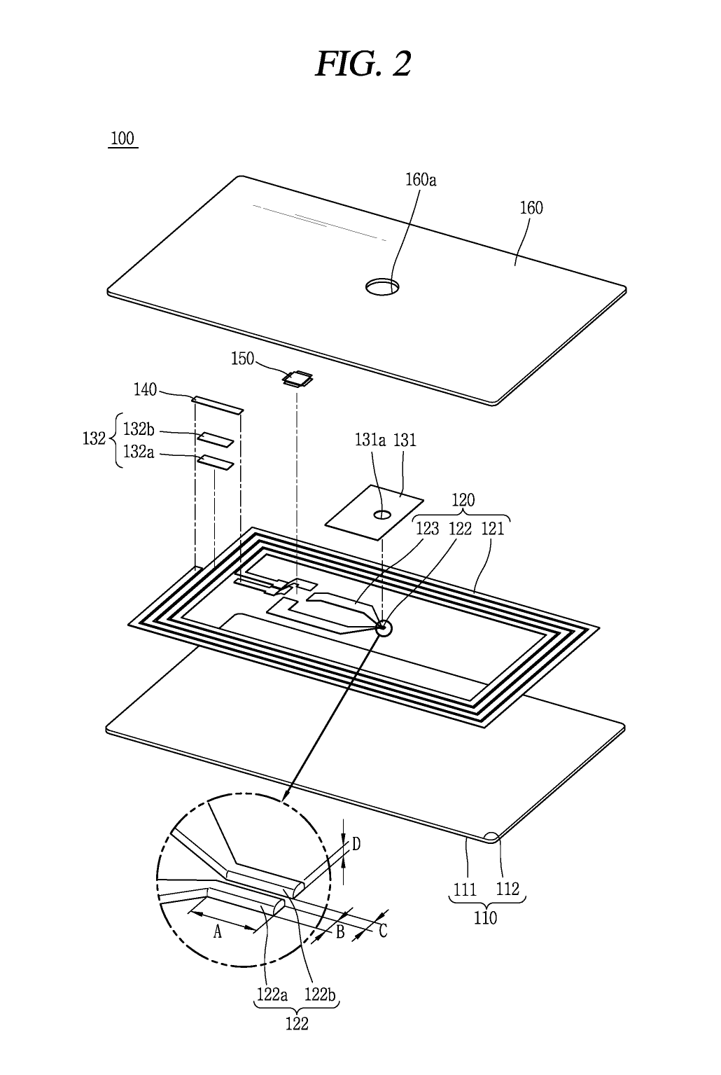

The substrate 110 may further include a silica layer 112. The silica layer 112 will be described later with reference to FIG. 2.

The conductive layer 120 is formed on one surface of the substrate 110 with a single layer. The conductive layer 120 may be formed by a printing process. The conductive layer 120 may include an antenna pattern 121, a circuit line 123 and a sensing electrode 122.

However, when the sensing unit is a field-effect transistor, the conductive layer 120 may include only the antenna pattern 121 and the circuit line 123. It will be described later with reference to FIG. 2.

The antenna pattern 121, sensing electrode 122 and circuit line 123 may be integrally formed. Being integrally formed denotes that the antenna pattern 121, sensing electrode 122 and circuit line 123 are not physically separated from one another. However, the antenna pattern 121, sensing electrode 122 and circuit line 123 may not be integrally formed. The antenna pattern 121, sensing electrode 122 and circuit line 123 are merely classified according to their functions.

The conductive layer 120 including the antenna pattern 121, sensing electrode 122 and circuit line 123 may be formed at the same time by one-time printing process, and the same layer may be formed on the substrate 110.

Forming the same layer has the same meaning as that of the antenna pattern 121, sensing electrode 122 and circuit line 123 being formed with the same height, respectively, on the substrate 110. Alternatively, forming the same layer may be also understood as the antenna pattern 121, sensing electrode 122 and circuit line 123 being formed at the same time by one-time printing process.

The conductive layer 120, including the antenna pattern 121, sensing electrode 122 and circuit line 123, may be formed at the same time by one-time printing process as they are formed of the same material.

Various methods such as a screen printing method, a gravure printing method, an ink-jet printing method or the like may be used for the printing process.

The antenna pattern 121 is formed to transmit and receive a wireless signal to and from an external device.

The external device denotes an electronic device having a wired/wireless communication function. The external device may include a portable phone, a smart phone, a desktop computer, a laptop computer, a digital broadcast terminal, a personal digital assistant (PDA), a portable multimedia player (PMP), a navigation device, a slate PC, a tablet PC, an ultrabook, and the like.

The antenna pattern 121 transmits a wireless signal to an external device or receives a wireless signal from the external device. The sensor 100 of the present disclosure does not have a power supply unit such as a battery. The sensor 100 is configured to generate direct-current power using the wireless signal received from the external device through the antenna pattern 121, and use the direct-current power for the operation of the sensor 100. Furthermore, the sensor 100 is configured to transmit a sensing result to the external device again through the antenna pattern 121.

The antenna pattern 121 is extended in a two-dimensional spiral shape, where the two-dimensional spiral shape denotes that the antenna pattern 121 is wound in a shape of being gradually closer to a central region of the substrate 110 from an edge of the substrate 110. It may also be described that the antenna pattern 121 is wound in a shape of being gradually closer to an edge of the substrate 110 from a central region of the substrate 110. However, according to the present disclosure, the two-dimensional spiral shape may not necessarily denote a curved line, and may include a straight line as illustrated in FIG. 1.

The antenna pattern 121 has a line width of 500-1,500 μm to have a high inductance. A distance between lines that form the two-dimensional spiral shape may be preferably set to 300-700 μm to have an appropriate capacitance component.

The antenna pattern 121 may be operated as a radiator of a near field communication (NFC) antenna. The NFC antenna indicates a communication device in which the exchange of information is carried out using the communication standard of 13.56 MHz.

The sensing electrode 122 is configured to cause an impedance change when in contact with a sensing target material.

When a wireless signal is received from an external device through the antenna pattern 121, the sensor 100 generates direct-current power using the wireless signal. Due to the generation of the direct-current power, the remaining portion of the sensor 100 is operated, and an alternating voltage is input to the sensing electrode 122. Accordingly, the sensing electrode 122 is operated only when a wireless signal is received through the antenna pattern 121.

When the sensing electrode 122 that receives an alternating voltage is brought into contact with a sensing target material, it generates an impedance change. The change of impedance generates a change of alternating voltage, and as a result, the sensor 100 may sense the salt content, sugar content of the sensing target material, the rancidity of edible fat and oil, and the like from the impedance change or the change of alternating voltage.

The sensing target material may be applicable to all materials that cause an impedance change. Foods having salt content, sugar content or the like, agricultural products in which pesticide residues exist, edible fat and oil capable of determining the rancidity are the examples of the sensing target material. The sensing target material may also include a liquid or gas.

The circuit line 123 electrically connects the antenna pattern 121 to the sensing electrode 122. Furthermore, the circuit line 123 is electrically connected to the device 150 for controlling the sensor 100. It should be understood that the circuit line 123 indicates all the remaining regions of the conductive layer 120 excluding the antenna pattern 121 and the sensing electrode 122.

The insulating layer 131, 132 is disposed on the conductive layer 120 to cover the conductive layer 120. The insulating layer 131, 132 may include the circuit insulating layer 131 and the antenna insulating layer 132. The circuit insulating layer 131 and the antenna insulating layer 132 may be formed at the same time by one-time printing process to form the same layer on the conductive layer 120.

According to an embodiment, forming the same layer has the same meaning as that of the circuit insulating layer 131 and antenna insulating layer 132 being formed with the same height, respectively, on the conductive layer 120. Alternatively, forming the same layer may also be understood as the circuit insulating layer 131 and the antenna insulating layer 132 being formed on the conductive layer 120 at the same time by one-time printing process. The circuit insulating layer 131 and the antenna insulating layer 132 may be formed at the same time by one-time printing process as they are formed of the same material.

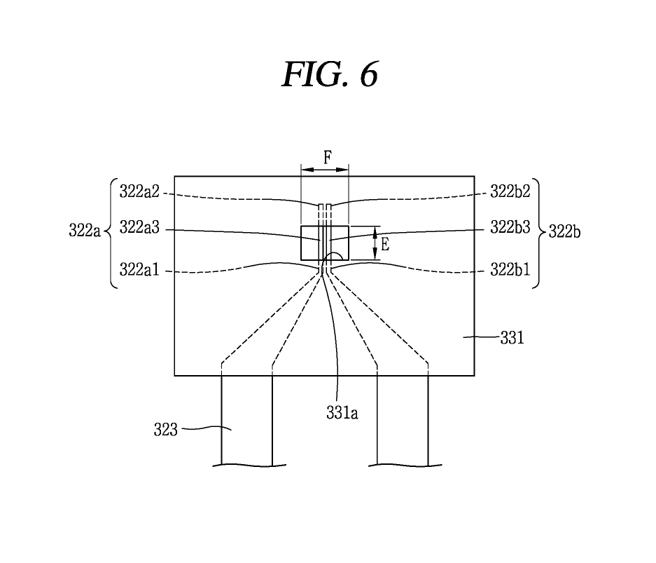

The circuit insulating layer 131 is disposed to cover at least part of the circuit line 123. The circuit insulating layer 131 may be disposed to partially cover the sensing electrode 122. The circuit insulating layer 131 has a flat plate shape. The circuit insulating layer 131 may include a window 131a (or hole) for exposing the sensing electrode 122. Even when a circumference of the circuit insulating layer 131 is disposed to cover the entire portion of the sensing electrode 122, the sensing electrode 122 may be exposed through the window 131a since the circuit insulating layer 131 includes the window 131a. The window 131a may set an exposure length of the sensing electrode 122, and it will be described later with reference to FIG. 6.

The circuit insulating layer 131 performs the role of preventing liquid to be brought into contact with the sensing electrode 122 from flowing into the region of the circuit line 123 and causing a malfunction of the sensor 100. The circuit insulating layer 131 is operated as a barrier to stop the flow of liquid since it has a predetermined height (a thickness direction of the sensor 100). Accordingly, the sensing target material may form a droplet within a region defined by the window 131a.

The circuit insulating layer 131 has surface energy (surface tension) larger than that of the substrate 110. If the circuit insulating layer 131 has surface energy smaller than that of the substrate 110, then the liquid in contact with the sensing electrode 122 may be spread without forming a droplet, and accordingly, sufficient sensing by the sensing electrode 122 may not be carried out. On the contrary, if the circuit insulating layer 131 has surface energy larger than that of the substrate 110, then the liquid may form a droplet, and sufficient sensing by the sensing electrode 122 may be carried out.

Referring to FIG. 1, the antenna pattern 121 has a two-dimensional spiral shape, thus causing a division between an inner side and an outer side at a boundary of the antenna pattern 121. The antenna bridge 140 has a configuration of connecting an outer side and an inner side of the antenna pattern 121 to each other, but the antenna bridge 140 may not be directly disposed on the antenna pattern 121. The antenna insulating layer 132 may perform the role of insulation between the antenna pattern 121 and the antenna bridge 140.

The antenna insulating layer 132 is disposed to cover any part of the antenna pattern 121. Referring to FIG. 1, it is seen that the antenna insulating layer 132 covers the left portion of the antenna pattern 121. Furthermore, the antenna insulating layer 132 is extended in a direction crossing the any part thereof. As the antenna insulating layer 132 is disposed between the antenna pattern 121 and the antenna bridge 140, insulation between the antenna pattern 121 and the antenna bridge 140 may be carried out.

The antenna insulating layer 132 may be formed with multiple layers, and each layer may be formed by a different printing process. When the antenna insulating layer 132 is formed with multiple layers, its bottom layer may be referred to as a first antenna insulating layer 132a (refer to FIG. 2), and its top layer may be referred to as a second antenna insulating layer 132b (refer to FIG. 2). As the circuit insulating layer 131 and the first antenna insulating layer 132a are formed during the one-time printing process, and the second antenna insulating layer 132b is additionally printed during a subsequent printing process, the circuit insulating layer 131 is formed with one layer, and the antenna insulating layer 132 is formed with two layers.

The antenna bridge 140 is disposed to cover the antenna insulating layer 132. One end of the antenna bridge 140 is connected to one end of the antenna pattern 121 located at an outer side of the substrate 110 and extended to cross the antenna pattern 121. The other end of the antenna bridge 140 is connected to one end of the circuit line 123 located at an inner side of the antenna pattern 121.

Alternatively, the antenna bridge 140 may connect an inner side and an outer side of the antenna pattern 121 to each other.

The device 150 is mounted on the substrate 110, and electrically connected to the circuit line 123.

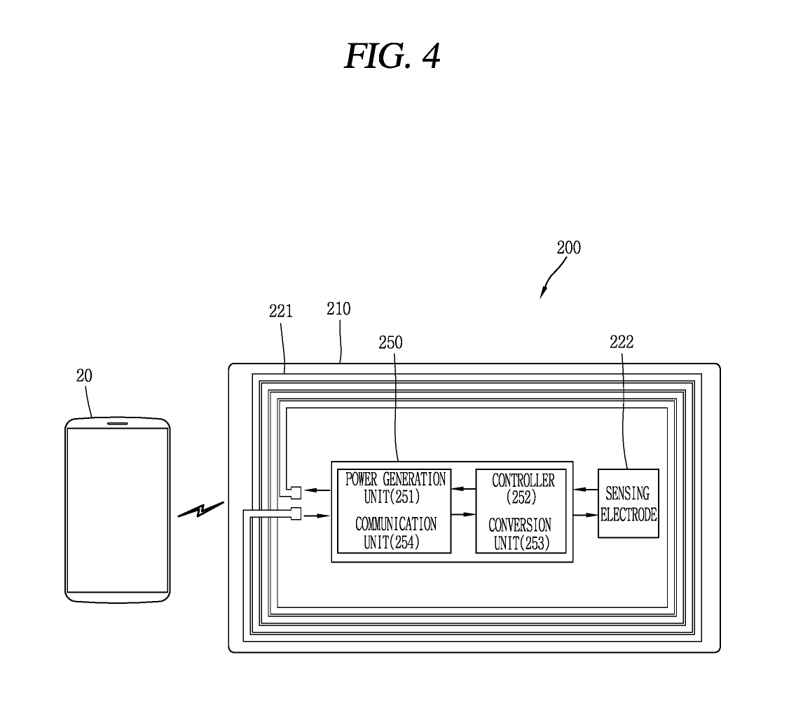

Various electrical and electronic configurations associated with the operation of the sensor 100 may be implemented by the device 150. The electrical and electronic configurations may indicate a power generation unit 251, a controller 252, a conversion unit 253, a communication unit 254, and the like which will be described later with reference to FIG. 4, for example.

A wireless signal received through the antenna pattern 121 is transmitted to the device 150 through the circuit line 123. Then, a voltage inputted to the sensing electrode 122 is inputted to the sensing electrode 122 through the circuit line 123.

Hereinafter, the multi-layer structure of the sensor 100 will be described with reference to FIG. 2.

FIG. 2 is an exploded perspective view illustrating the sensor 100 according to the present disclosure. It is illustrated in FIG. 2 that the antenna pattern 121, sensing electrode 122 and circuit line 123 form the same layer, and the circuit insulating layer 131 and antenna insulating layer 132 form the same layer.

The substrate 110 may include a plastic layer 111 and a silica layer 112.

The plastic layer 111 is formed of plastic (e.g., polymer compound or synthetic resin) having flexibility. The plastic may include at least one selected from a group consisting of polyethylene terephthalate (PET), polyimide (PI), polystyrene (PS) and polyethylene naphthalate (PEN).

The silica layer 112 is coated on one surface of the plastic layer 111. The silica layer 112 is formed between the plastic layer 111 and the conductive layer 120. The silica layer 112 is provided for the rapid spreading of sensing target solution, the stabilization of sensing target solution, and the adhesion strength enhancement of the conductive layer 120. The silica layer 112 may have a thickness of several or several tens of nanometers.

The conductive layer 120 is formed with a single layer. Furthermore, the antenna pattern 121, sensing electrode 122 and circuit line 123 may be integrally formed, and the antenna pattern 121, sensing electrode 122 and circuit line 123 may form the same layer (e.g., a single layer). The antenna pattern 121, sensing electrode 122 and circuit line 123 may be integrally formed, where being integrally formed denotes that the antenna pattern 121, sensing electrode 122 and circuit line 123 are not physically separated from one another. However, the antenna pattern 121, sensing electrode 122 and circuit line 123 may not be integrally formed.

The conductive layer 120 being formed with a single layer has substantially the same meaning as that of the antenna pattern 121, sensing electrode 122 and circuit line 123 forming the same layer. It is because the antenna pattern 121, sensing electrode 122 and circuit line 123 are formed of the same material.

The conductive layer 120 is formed by a printing process. If the antenna pattern 121, sensing electrode 122 and circuit line 123 are formed by a plurality of printing processes, respectively, then the antenna pattern 121, sensing electrode 122 and circuit line 123 may form different layers. As a result, the conductive layer 120 may be formed with a multi-layer rather than a single layer.

However, if the antenna pattern 121, sensing electrode 122 and circuit line 123 are formed of the same material as in the present disclosure, then the conductive layer 120 with a single layer may be formed by one-time printing process. Furthermore, the conductive layer 120 formed as described above may be divided into three portions according to a function thereof, and thus the antenna pattern 121, sensing electrode 122 and circuit line 123 may be divided into a first portion, a second portion and a third portion, respectively.

If the antenna pattern 121, sensing electrode 122 and circuit line 123 form the same layer (e.g., one layer), then the sensor 100 may have several advantages compared to the related art.

First, the thickness of the sensor 100 may be reduced, and thus the sensor 100 may be further miniaturized compared to the related art. When the sensor 100 is miniaturized, it may be possible to enhance the portability of the sensor 100.

Furthermore, the fabrication process of the sensor 100 may be further simplified, and thus the sensor 100 may be fabricated at a lower cost compared to the related art.

Finally, no process error may occur between the antenna pattern 121, sensing electrode 122 and circuit line 123. If the antenna pattern 121, sensing electrode 122 and circuit line 123 are sequentially formed by different printing processes, the sensor 100 may not be fabricated with the original design due to a process error (e.g., particularly, alignment error). The process error causes a problem during mass production. However, if the antenna pattern 121, sensing electrode 122 and circuit line 123 are formed with one-time printing process, such a problem may be solved.

The sensing electrode 122 may include a first electrode 122a and a second electrode 122b. The first electrode 122a and second electrode 122b are extended in parallel from the circuit line 123, respectively, and a gap exists between the first electrode 122a and the second electrode 122b.

Hereinafter, the structure or specification of the sensing electrode 122, specifically, a length, a width, a gap and a height of the sensing electrode 122 will be described. Referring to an enlarged portion of the sensing electrode 122 in FIG. 2, the length, width, gap and height of the entire sensing electrode 122 are shown as A, B, C and D, respectively.

The length (A) of the entire sensing electrode 122 may be 400-5,000 μm, and an exposure length (A′, a length corresponding to “E” in FIG. 6) of the sensing electrode 122 exposed through the window 131a may be 50-5,000 μm. For reference, the entire portion of the sensing electrode 122 illustrated in FIG. 1 is exposed. Accordingly, the exposure length (A′) of the sensing electrode 122 exposed through the window 131a is the same as the length (A) of the entire sensing electrode 122. However, as will be described later in FIG. 6, the exposure length of the sensing electrode 122 is not always the same as the length of the entire sensing electrode 122.

The exposure length (A′) of the sensing electrode 122 exposed through the window 131a has an effect on the resolution of the sensor 100, printing reproducibility according to mass production, and the reliability of the sensor 100. As the exposure length of the sensing electrode 122 exposed through the window 131a is smaller, the resolution of the sensor 100 may be enhanced.

For example, when the exposure length (A) of the sensing electrode 122 exposed through the window 131a is formed at about 300-500 μm, the resolution of the sensor 100 may be greatly enhanced. However, it may be difficult to accurately locate and print the window 131a at the sensing electrode 122 during the fabrication process of the sensor 100. Accordingly, the exposure length (A′) of the sensing electrode 122 may be preferably formed larger than 300 μm. Furthermore, when the length (A) of the entire sensing electrode 122 is smaller than 400 μm, the exposure length (A′) of the sensing electrode 122 exposed through the window may not be constant due to the occurrence of a location error of the window 131a according to mass production. Furthermore, when the length (A) of the entire sensing electrode 122 is smaller than 400 μm, an impedance increases as a number of uses of the sensor 100 increases, thus reducing the durability and reliability of the sensing electrode 122.

Accordingly, the length (A) of the entire sensing electrode 122 may preferably be larger than 400 μm. Furthermore, the exposure length of the sensing electrode 122 may be preferably smaller than 2,000 μm to secure the resolution of the sensor 100. In addition, the length (A) of the entire sensing electrode 122 may be formed up to 5,000 μm.

The width (B) of the sensing electrode 122 may be formed at 50-1,000 μm. As the width (B) of the sensing electrode 122 decreases, the resolution of the sensor 100 may be enhanced. However, an extremely small width (B) of the sensing electrode 122 may destabilize the printing process of the conductive layer 120. Accordingly, the width (B) of the sensing electrode 122 may be preferably 50-200 μm for a stable printing process.

A distance (C) between the first electrode 122a and the second electrode 122b may be 50-3,000 μm. However, an extremely large distance (C) may cause an insufficient operation of the sensor 100 when an amount of the sensing target liquid is insufficient. The sensing target liquid should form a droplet to be brought into contact with both the first electrode 122a and second electrode 122b because the liquid might be brought into contact with only either one of the electrodes when a distance between the first electrode 122a and the second electrode 122b is extremely large. In consideration of this, a distance (C) between the first electrode 122a and the second electrode 122b may be preferably 900-1,500 μm.

A height (D) (e.g., thickness direction of the sensor 100) of the sensing electrode 122 may be 700 nm-15 μm. The height (D) of the sensing electrode 122 may have an effect on the thickness of the sensor 100 and the durability and reliability of the sensing electrode 122. When the height (D) of the sensing electrode 122 is less than 700 nm, a problem of losing the sensing electrode 122 may occur due to the repetitiveness of sensing. Furthermore, in consideration of the limitation of the printing process and the thickness increase of the sensor 100, the height (D) of the sensing electrode 122 may be preferably less than 15 μm. Since the sensing electrode 122 is a portion of the conductive layer 120, the height (D) of the sensing electrode 122 immediately denotes a height of the conductive layer 120.

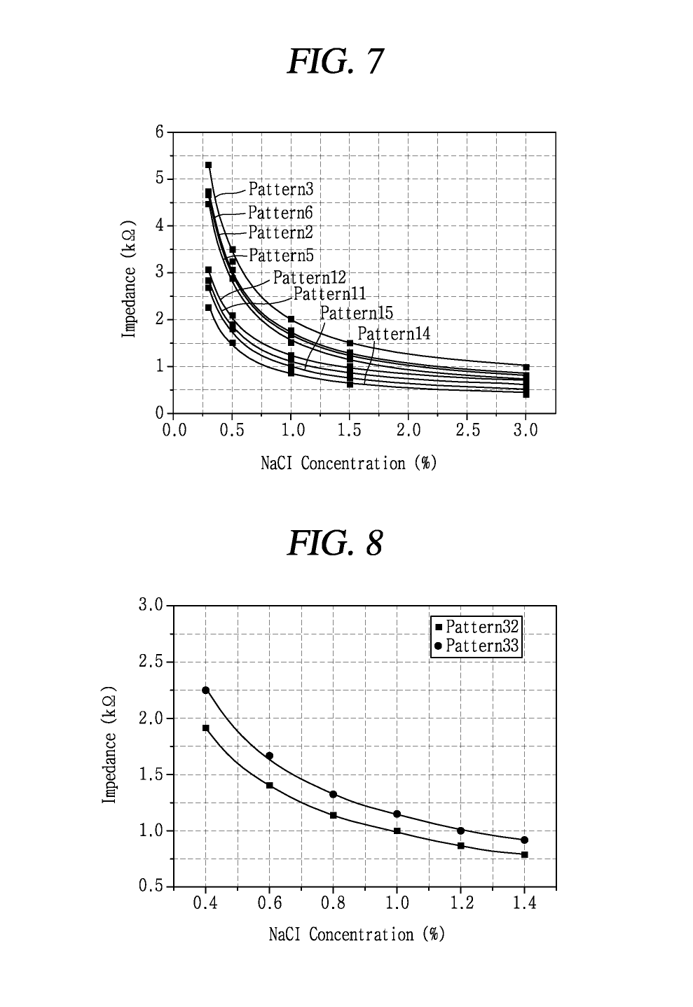

A relationship between the structure of the sensing electrode 122 and the resolution of the sensor 100 will be described again with reference to the graphs of FIGS. 7 and 8.

The sensor 100 may include a protective layer 160. The protective layer 160 is formed of an electrical insulating material. The protective layer 160 is disposed to face one surface of the substrate 110, and configured to cover all the constituent elements of the sensor 100. The protective layer 160 may include a hole 160a, and the sensing electrode 122 may be exposed through the hole 160a.

In order to help the user's understanding, an indicator indicating an inlet position of sensing target liquid may be printed on an outer surface of the protective layer 160.

The circuit insulating layer 131 and first antenna insulating layer 132a being formed by one-time printing process is similar to the antenna pattern 121, sensing electrode 122 and circuit line 123 being formed by one-time printing process. Accordingly, the circuit insulating layer 131 and first antenna insulating layer 132a have the same advantage as that of the antenna pattern 121, sensing electrode 122 and circuit line 123.

It is seen from FIG. 2 that second antenna insulating layer 132b is disposed on the first antenna insulating layer 132a.

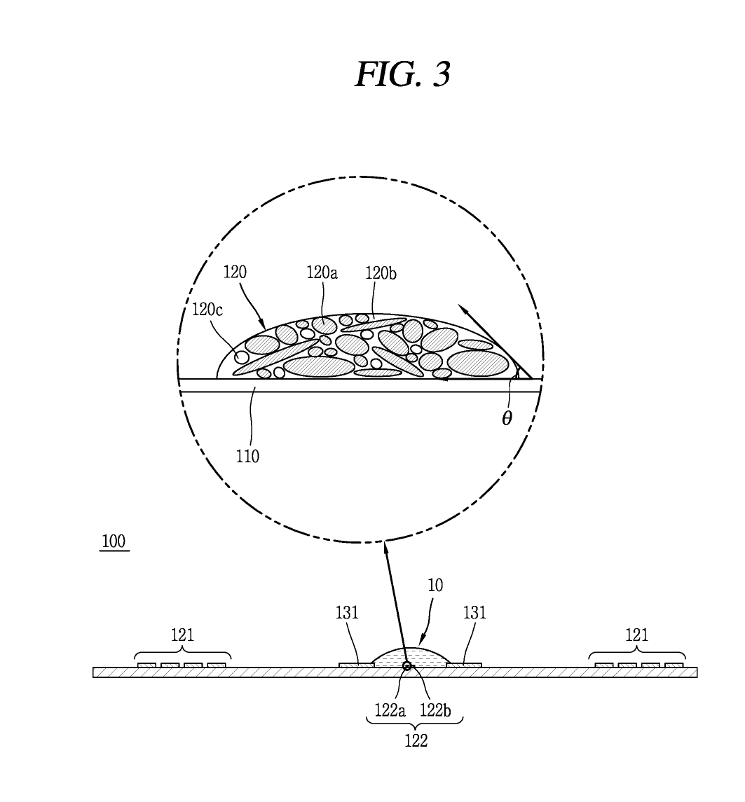

FIG. 3 is a cross-sectional view taken along line A-A in FIG. 1. The circuit insulating layer 131 may have a height of 80 nm-30 μm. The circuit insulating layer 131 has a height higher than that of the sensing electrode 122. In other words, the circuit insulating layer 131 is printed at a thickness larger than the height of the sensing electrode 122. The circuit insulating layer 131 performs the role of a barrier for suppressing the flow of a droplet. Accordingly, when the height of the circuit insulating layer 131 is lower than that of the sensing electrode 122, liquid in contact with the sensing electrode 122 passes over the circuit insulating layer 131. Accordingly, the circuit insulating layer 131 may preferably have a height higher than that of the sensing electrode 122.

Since the circuit insulating layer 131 and the antenna insulating layer 132 illustrated in FIGS. 1 and 2 are formed of the same material to form the same layer, and formed by one-time printing process, the antenna insulating layer 132 may be also expected to have a height of 800 nm-30 μm. However, the antenna insulating layer 132 may be formed with multiple layers, and in this case, it may be expected that the height of each layer is 800 nm-30 μm.

FIG. 3 illustrate a state in which a sensing target material is in contact with the sensing electrode 122. It is seen that the sensing target material in a liquid phase forms a droplet, and is confined within the window 131a by the circuit insulating layer 131.

Subsequently, the following description will be described with reference to an enlarged portion of the conductive layer 120.

The conductive layer 120 may include solid particles 120a and organic substances 120b.

The solid particles 120a may be formed of at least one of silver (Ag), copper (Cu) and aluminium (Al).

Among the related arts, there is a technology in which a precious metal such as platinum (Pt) or gold (Au) is used as a material of the sensing electrode 122, and the precious metal is deposited or calcinated on the substrate 110 at high temperatures. According to the related art, the circuit line 123 and sensing electrode 122 are formed on different layers. However, the precious metal causes a cost increase of the sensor 100. Furthermore, the circuit line 123 and sensing electrode 122 being formed on different layers denotes that it does not have an advantage of the present disclosure described in FIG. 2.

On the contrary, silver (Ag), copper (Cu) and aluminium (Al) is cheaper than precious metals, and thus the sensor 100 of the present disclosure has an advantage of reducing the unit cost of the sensor 100 compared to the related art. However, silver (Ag), copper (Cu) and aluminium (Al) has a low standard reduction potential compared precious metals such as platinum (Pt) or gold (Au). Accordingly, the sensing electrode 122 may be modified by oxidation or corrosion during the sensing process.

According to the present disclosure, a measurement voltage of the sensor 100 is limited to 0.1-4 V to suppress the oxidation or corrosion of the conductive layer 120. Furthermore, the present disclosure has solved the oxidation or corrosion problem of the conductive layer 120 through the structure of the sensing electrode 122 and the material optimization of the conductive layer 120. The structure of the sensing electrode 122 has been described in FIG. 2. The material optimization of the conductive layer 120 relates to the content of the solid particles 120a and organic substances 120b.

Furthermore, the solid particle 120a has a spherical or flake shape. For the conductive layer 120 illustrated in FIG. 3, the shapes other than the spherical shapes may be all referred to as flake shapes. The solid particle 120a in the flake shape may have a relatively higher conductivity than that of the spherical shape.

The solid particle 120a may have a size of several tens of nm-20 μm to secure the reaction surface area. The sensing electrode 122 reacts with a sensing target material and causes an impedance change, and there exist a capacitance component and a resistance component in the impedance. When the reaction surface area of the solid particle 120a increases, it may be possible to suppress the oxidation or corrosion of the sensing electrode 122 due to the reaction as well as extend the lifespan of the sensor 100.

The organic substance 120b supports the solid particle 120a. The solid particle 120a exposed to an outside of the organic substance 120b may be lost subsequent to sensing, but the solid particle 120a existing within the organic substance 120b may be protected by the organic substance 120b. Accordingly, the organic substance 120b performs the role of enhancing the durability and reliability of the sensor 100.

The organic substance 120b may include at least one organic substance selected from a group consisting of polyethylene oxide (PEO) series, oleic acid series, acrylate series, acetate series and epoxy series.

The conductive layer 120 has a pore 120c. The pore 120c may have a size of several nm-several tens of μm. When the conductive layer 120 has the pore 120c, it may not be easily damaged by a repetitive mechanical deformation, thereby enhancing the reliability of the sensor 100. As illustrated in FIG. 1, the substrate 110 may also have flexibility, and thus the conductive layer 120 may preferably have the pore 120c.

The conductive layer 120 and substrate 110 form a contact angle (θ) at an acute angle. The contact angle (θ) described in the present disclosure is illustrated in FIG. 3. A cross section of the sensing electrode 122 formed with the existing vacuum deposition method has a rectangular shape to form a contact angle (θ) at an obtuse angle with substrate 110. The sensing electrode 122 forming the contact angle (θ) at an obtuse angle may be easily separated from the substrate 110 by friction or bending. However, as in the present disclosure, the sensing electrode 122 forming the contact angle (θ) at an acute angle with the substrate 110 may have an excellent resistance to friction or bending, and maintain durability without an additional protective layer.

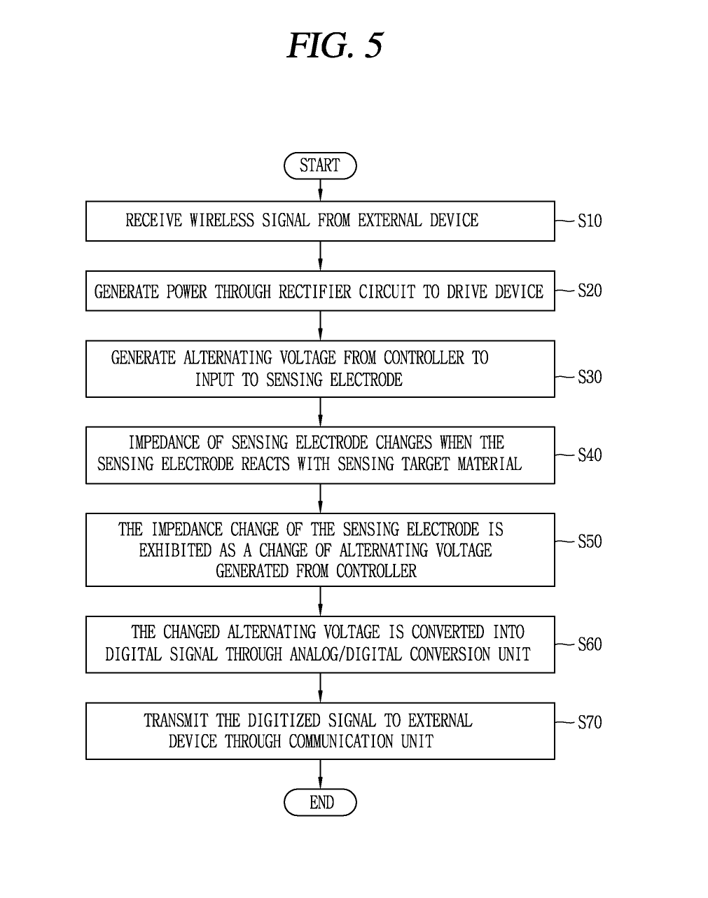

FIG. 4 is a conceptual view illustrating an interrelated operation between an external device and a sensor 200. FIG. 5 is a flow chart illustrating a method of measuring the state of a measurement target material using the sensor 200.

In FIG. 4, a circuit unit 250 includes a power generation unit 251, a controller 252, a conversion unit 253, and a communication unit 254. The circuit unit, which includes the power generation unit 251, controller 252, conversion unit 253 and communication unit 254, may be implemented by the device 150 described in FIG. 1.

Referring to FIG. 4, an external device 20 includes a mobile terminal having a wireless communication function and a display.

First, the sensor 200 receives a wireless signal from the external device 20 through the antenna pattern 210 (S10).

Subsequently, the sensor 200 generates direct-current power through the power generation unit 251 (for example, a rectifier circuit of NFC Tag IC) to drive the circuit unit 250 (S20). As described above, the sensor 200 of the present disclosure generates direct-current power using a wireless signal received from an external device to operate the controller 252, conversion unit 253, communication unit 254 and sensing electrode 222, and the sensor 200 is not configured to supply power by itself.

The controller 252 is driven by receiving direct-current power. The controller 252 generates an alternating voltage (for example, 10 KHz, 1.2 V) fed to the sensing electrode 222 (S30).

When the sensing electrode 222 reacts with a sensing target material, the sensing electrode 222 causes an impedance change (S40). Then, the impedance change of the sensing electrode 222 is shown as a change of alternating voltage generated from the controller 252 (S50). The sensing target materials may be classified according to a range of an output value.

The changed alternating voltage is converted into a digital signal (S60). The conversion unit 253 converts the changed alternating voltage based on the impedance change of the sensing electrode 222 into the digital signal. Then, the communication unit 254 (for example, NFC Tag IC) transmits the digital signal to the external device through the antenna pattern 221 (S70).

The external device 20 receives the digital signal from the sensor 200 to generate, store and manage information. Furthermore, the external device 20 may display information through the display.

As described above, the sensor 200 is driven by generating direct-current power from a signal received through the antenna pattern 221. Furthermore, the sensor 200 is configured to transmit a signal generated by the measurement of the sensing electrode 222 to the external device 20. Accordingly, the sensor 200 is equipped with neither a power supply nor a display since it is able to communicate with the external device 20.

FIG. 6 is a conceptual view illustrating the structure of a circuit line 323, a sensing electrode 322 and a circuit insulating layer 331 that are advantageous for mass production.

There exists a process error in mass production. Accordingly, the final product may not always be produced as designed. In particular, as a size of the product decreases, the probability of occurrence for a process error increases. In consideration of such a process error, a ratio of a number of products made as designed to a total number of products made is shown as a yield.

The sensor of the present disclosure is formed by multiple printing processes, and a heat-drying and curing process. The probability of occurrence for a process error is very low during one-time printing process, but when the printing process for each layer is repeated, a process error (e.g.., particularly, alignment error) may occur, and the process error may also occur due to the contraction of the substrate (for example, reference numeral 110 in FIG. 1). In particular, the substrate 110 is largely contracted in a lengthwise direction.

In particular, the resolution of the sensor may be determined according to the exposure length of a first and a second sensing electrode 322a, 322b. However, the entire length and exposure length of the first and the second sensing electrode 322a, 322b is very small (e.g., in the 5,000-μm range). Accordingly, during the mass production of the sensor, the probability of occurrence for a process error to the exposure length of the sensor is high. Accordingly, a structure capable of minimizing the process error should be provided to secure the resolution of the sensor.

The first and the second sensing electrode 322a, 322b, circuit line 323 and circuit insulating layer 331 are all formed by a printing process, and the circuit insulating layer 331 is disposed to cover the first and the second sensing electrode 322a, 322b and circuit line 323. Accordingly, the circuit insulating layer 331 is formed subsequent to printing the first and the second sensing electrode 322a, 322b and circuit line 323. Accordingly, a process error may occur during the repetitive printing processes and the heat-drying process, where there may be a case where the first and the second sensing electrode 322a, 322b are exposed to be different from the original design or a case where the first and the second sensing electrode 322a, 322b are not exposed at all.

FIG. 6 illustrates the structure of a sensor 300 for solving such a problem.

The first and the second sensing electrode 322a, 322b may be divided into three portions according to a lengthwise direction. The first and the second sensing electrode 322a, 322b may include a first end portion 322a1, 322b1 and a second end portion 322a2, 322b2 in a lengthwise direction. Furthermore, a region between the first end portion 322a1, 322b1 and the second end portion 322a2, 322b2 may be referred to as a central portion 322a3, 322b3. The circuit insulating layer 331 is formed to cover the first end portion 322a1, 322b1 and second end portion 322a2, 322b2, and a window 331 a is formed to expose a region (central portion) 322a3, 322b3 between the first end portion 322a1, 322b1 and the second end portion 322a2, 322b2.

The sensing electrode 322a, 322b is formed to be larger than the length (E) of the window 331a. The window 331a controls the exposure length of the sensing electrode 322. For example, when the window 331a is 500-2,000 μm in length, the exposure length of the sensing electrode 322 exposed through the window 331a is always determined as 500-2,000 μm as the window 331a exposes the central portion 322a3, 322b3. Even if the positions of the sensing electrode 322 and circuit insulating layer 331 are exchanged somewhat, the exposure length of the sensing electrode 322 exposed through the window 331a may be maintained in the range of 500-2,000 μm. Through the foregoing structure, the sensor of the present disclosure may obtain a high yield even though there is a process error in mass production.

It is preferable that a width (F) of the window 331 a is wider than a distance between the first electrode 322a and the second electrode 322b, but it does not exceed 5,000 μm. It is because when the width of the window 331a is extremely large, liquid to be brought into contact with the sensing electrode 322a, 322b may be spread out without forming a droplet.

FIGS. 7 and 8 are graphs experimentally illustrating a relationship between the structure and resolution of the sensing electrode.

A horizontal axis of the graph indicates a concentration of sodium chloride, and a vertical axis thereof indicates an impedance. Each pattern has a different structure of the sensing electrode. The structure of the sensing electrode denotes the length and width of the sensing electrode, and the distance between two electrodes. The structure of the sensing electrode in each pattern is summarized in Table 1.

TABLE 1

| Pattern | Length (μm) | Width (μm) | Gap (μm) |
|---|---|---|---|
| 2 | 500 | 100 | 200 |
| 3 | 500 | 100 | 300 |
| 5 | 500 | 200 | 200 |
| 6 | 500 | 200 | 300 |
| 11 | 1,000 | 100 | 200 |
| 12 | 1,000 | 100 | 300 |
| 14 | 1,000 | 200 | 200 |
| 15 | 1,000 | 200 | 300 |
| 32 | 1,250 | 100 | 900 |
| 33 | 1,600 | 100 | 900 |

A steep slope of the graph denotes the sensing electrode sensitively reacting with a small change of concentration of sodium chloride, thus having a higher resolution.

Referring to the graphs of FIGS. 7 and 8, the resolution is enhanced as the length of the sensing electrode exposed through the window is shorter, the width of the sensing electrode is narrower, and a gap between two electrodes is larger. However, if the sensing electrode is merely designed only for the purpose of enhancing the resolution, then it may cause a problem such as durability, reliability and the like. Accordingly, the structure of the sensing electrode should be designed in consideration of resolution, durability, reliability, and the like, and an appropriate structure of the sensing electrode has been previously described in the above.

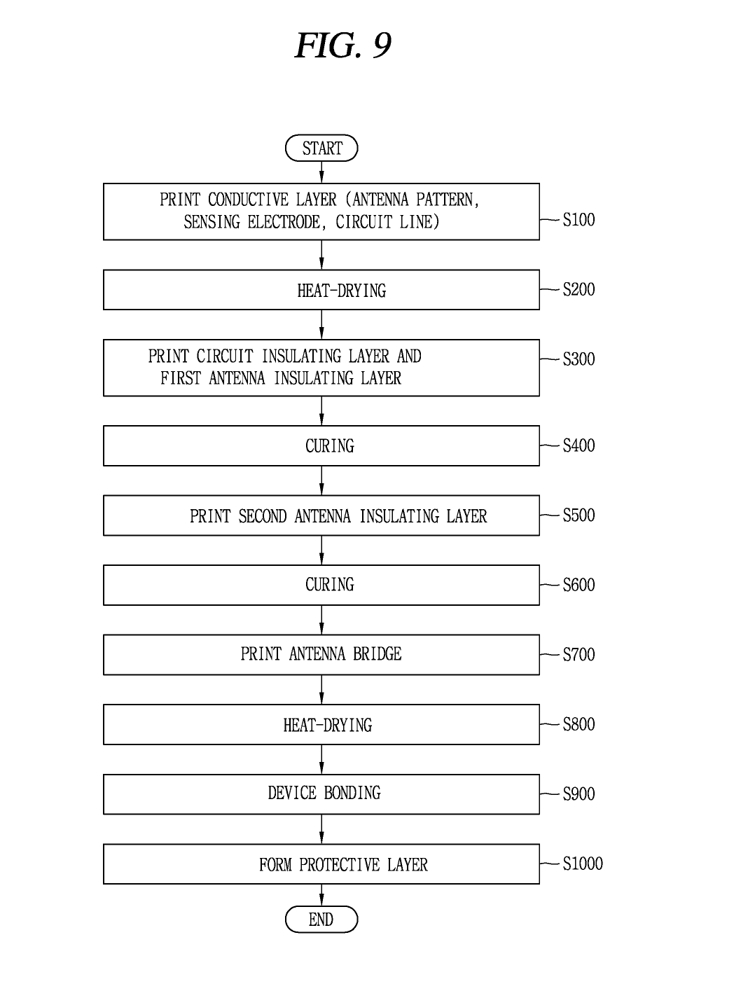

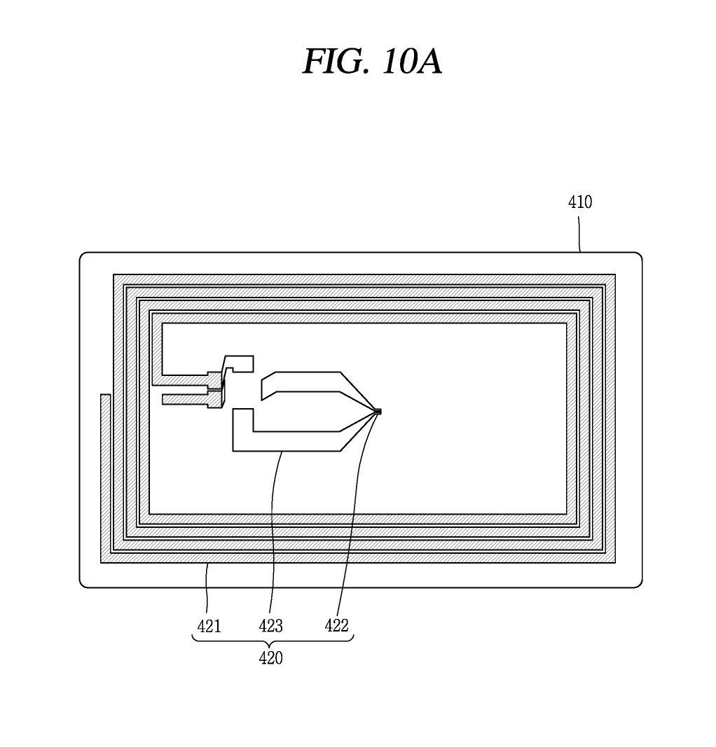

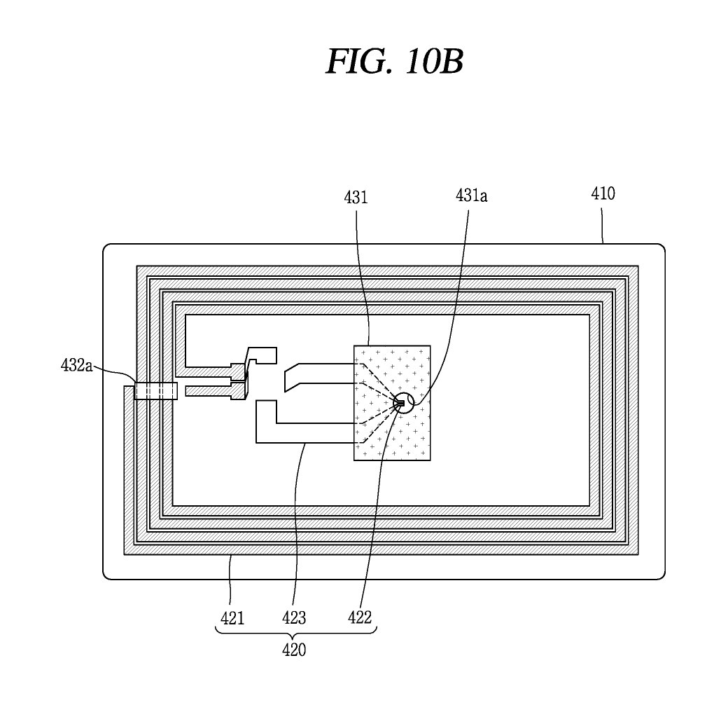

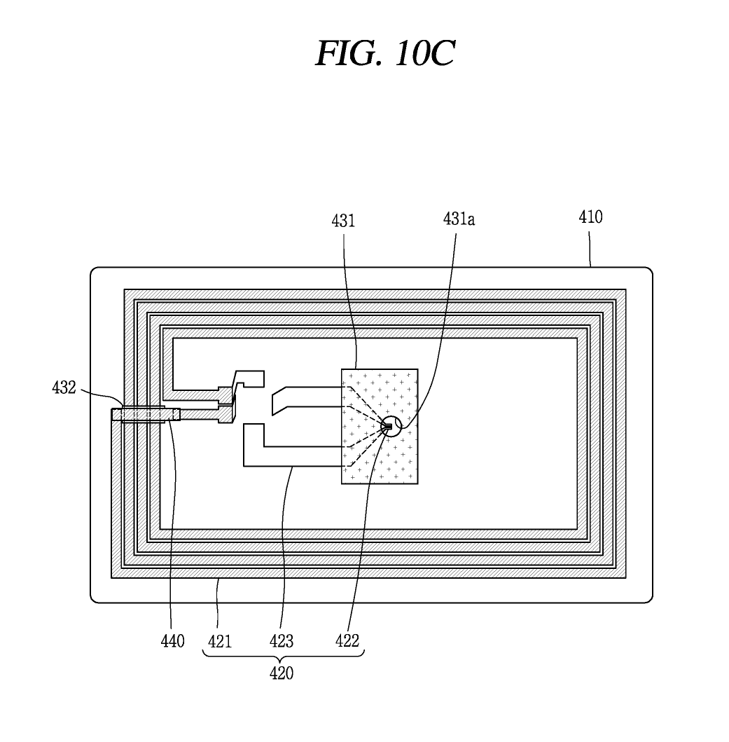

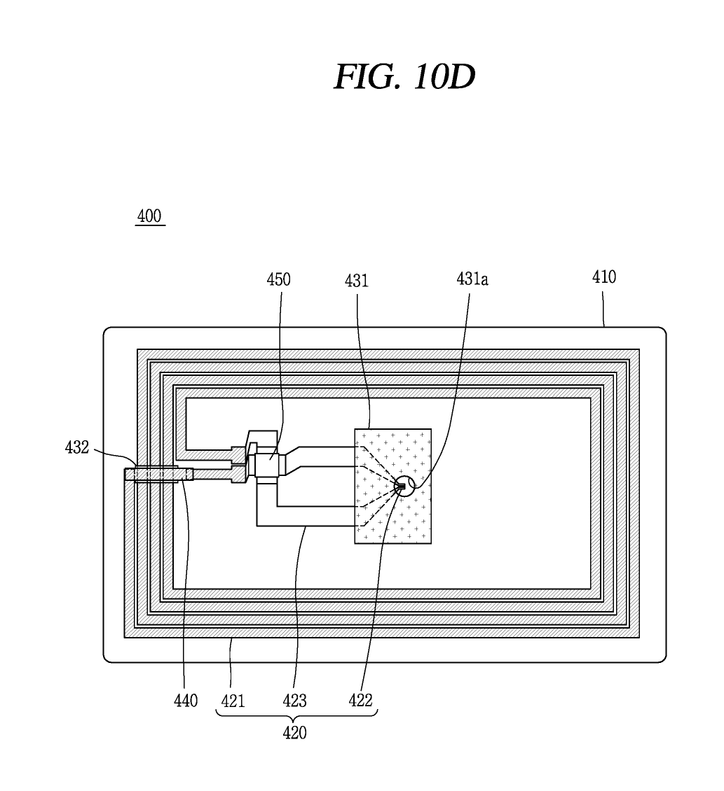

FIG. 9 is a flow chart illustrating a method of fabricating a sensor. FIGS. 10A through 10D are conceptual views partially illustrating a fabrication process of a sensor according to the fabrication method of FIG. 9.

First, a conductive layer is printed on a substrate through a printing process (S100). The conductive layer may include an antenna pattern, a sensing electrode and a circuit line. Accordingly, the antenna pattern, sensing electrode and circuit line are formed on the substrate at the same time through a process of printing the conductive layer on the substrate. The antenna pattern, sensing electrode and circuit line should be all formed of the same material to form the antenna pattern, sensing electrode and circuit line at the same time.

The advantage of forming the antenna pattern, sensing electrode and circuit line at the same time and forming one layer has been previously described in the above.

The printing process of the conductive layer uses ink powder or paste.

The composition of ink powder or paste may include 40-70 weight percent of solid particles and 30-60 weight percent of an organic substance containing a solvent. The composition is a composition capable of solving the oxidation and corrosion problem of the sensing electrode.

The solid particles are formed of at least one of silver (Ag), copper (Cu) and aluminium (Al). The solid particles have a spherical shape or flake shape.

The organic substance may include at least one selected from a group consisting of polyethylene oxide (PEO) series, oleic acid series, acrylate series, acetate series and epoxy series.

The solvent may include at least one selected from a group consisting of acetone, allyl alcohol, acetic acid, acetol, methyl alcohol and benzene.

The process of printing the conductive layer may use any one of screen, offset, gravure, but the present disclosure may not be necessarily limited to this. All printing processes capable of forming the antenna pattern, sensing electrode and circuit line at the same time may be used for the present disclosure.

FIG. 10A illustrates a substrate 410 and a conductive layer 420 subsequent to printing the conductive layer. Reference numerals 421, 422 and 423 indicate an antenna pattern, a sensing electrode and a circuit line, respectively.

Referring to FIG. 9 again, subsequently, the conductive layer is heat-dried (S200). The heat-drying may be carried out at temperatures of 80-200 ° C. The solvent may be evaporated during the heat-drying process. A process error due to contraction that can occur during the heat-drying process and a structure of the sensing electrode for solving the process error has been described previously.

In order to achieve a low-temperature process below 200° C., the solid particles may preferably have a size of several tens of nm-20 μm in a powder form.

Next, the circuit insulating layer and first antenna insulating layer are printed (S300).

As the circuit insulating layer and first antenna insulating layer are also formed of the same material, they may be formed at the same time by one-time printing process. The circuit insulating layer and first antenna insulating layer form the same layer on a conductive layer.

FIG. 10B illustrates a substrate 410, a conductive layer 420, an circuit insulating layer 431 and a first antenna insulating layer 432a subsequent to printing the circuit insulating layer 431 and first antenna insulating layer 432a.

Referring to FIG. 9 again, the circuit insulating layer and first antenna insulating layer are cured (S400). The curing may be carried out by ultraviolet light (UV). Subsequently, a second antenna insulating layer is printed on the first antenna insulating layer (S500). Then, the second antenna insulating layer is cured (S600). Similarly, the curing may be carried out using ultraviolet light (UV).

The second antenna insulating layer is to achieve more secure insulation. Accordingly, if it is sufficiently insulated by the first antenna insulating layer, then the process of printing the second antenna insulating layer and the process of curing it may be omitted. On the contrary, if it is not sufficiently insulated by the first antenna insulating layer and second antenna insulating layer, then the process of additionally printing and curing a third antenna insulating layer on the second antenna insulating layer may be added thereto.

Next, an antenna bridge is printed on the second antenna insulating layer (S700). The antenna bridge may be formed of the same material as that of the conductive layer. FIG. 10C illustrates a substrate 410, a conductive layer 420, a circuit insulating layer 431, a first antenna insulating layer 432 and an antenna bridge 440.

Referring to FIG. 9, again, the antenna bridge is heat-dried (S800). The process of heat-drying the antenna bridge may be substantially carried out similarly to the process of heat-drying the conductive layer.

Next, a device is bonded to the substrate (S900). The device is electrically connected to a circuit line. FIG. 10D illustrates a sensor 400 subsequent to bonding the device 450.

Finally, a protective layer for covering all the constituent elements of the sensor is formed (S1000).

Next, a case where the sensing unit is the field-effect transistor will be described.

Prior to describing the case, the field-effect transistor will be described in detail.

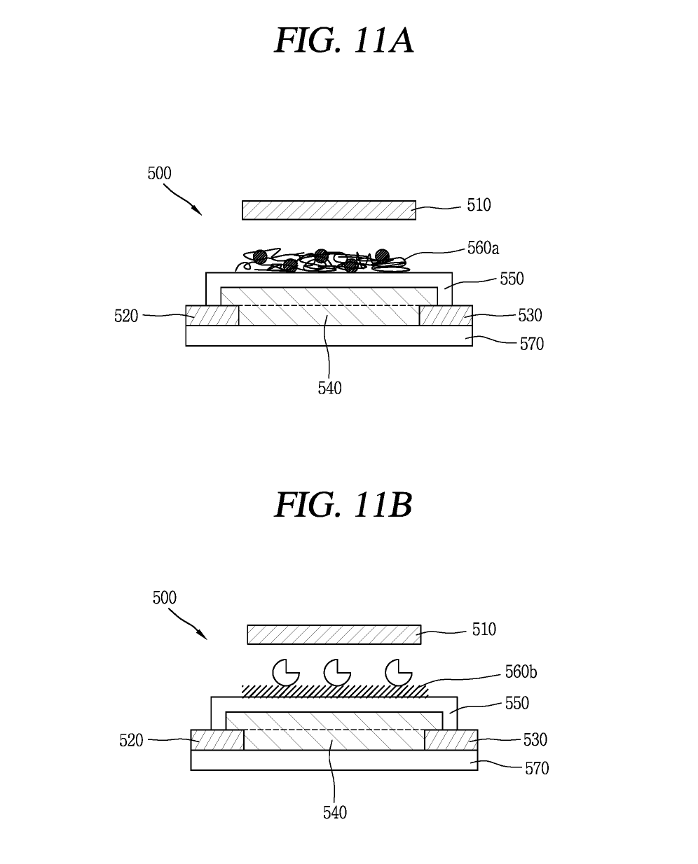

FIGS. 11A and 11B are conceptual views illustrating a field-effect transistor according to the present disclosure.

Hereinafter, the field-effect transistor according to the present disclosure will be described with reference to FIGS. 11A and 11B.

The field-effect transistor may be formed on a substrate, and may include a gate 510, a source 520 and a drain 530 electrode. Specifically, the field-effect transistor according to the present disclosure is a symmetrical device, and there is no structural difference in the source and drain electrodes. Thus when a voltage is applied to both electrodes, the source and drain electrodes are determined according to a voltage value applied to the electrodes.

On the other hand, when a voltage is applied to the gate and source electrodes, a channel layer 540 is formed between the source and drain electrodes. It is similar to a previously known field-effect transistor, and thus the description thereof will be omitted.

On the other hand, according to a field-effect transistor according to the present disclosure, a current flowing along the channel layer varies by a contact target material. To this end, the field-effect transistor according to the present disclosure may include an insulating layer 550 and a sensing layer 560.

Specifically, the channel layer 540 is located between the source 520 and drain 530 electrodes, the insulating layer 550 is located on the channel layer 540, and the sensing layer 560 is located on the insulating layer 550.

Here, the sensing layer 560 is separated from the gate 510 electrode by a predetermined distance. Accordingly, the contact target material may be introduced into the sensing layer 560. A distance between the sensing layer 560 and the gate 510 may be 0.01-10 mm.

Hereinafter, the present disclosure will be described in detail along with the constituent elements thereof.

In a field-effect transistor 500 according to the present disclosure, a voltage applied to the channel layer 540 varies due to a contact target material. Accordingly, a current flowing along the channel layer 540 varies.

Specifically, the present disclosure provides a field-effect transistor capable of detecting glucose, namely, urine glucose, among the components of urine. In other words, the contact target material may be glucose.

The sensing layer may include an enzyme capable of oxidizing glucose. Specifically, the enzyme may be at least one of glucose oxidase (GOX) or glucose dehydrogenase (GDH). As shown in the following Chemical Formula 1, pH decreases as glucose is oxidized into gluconate.

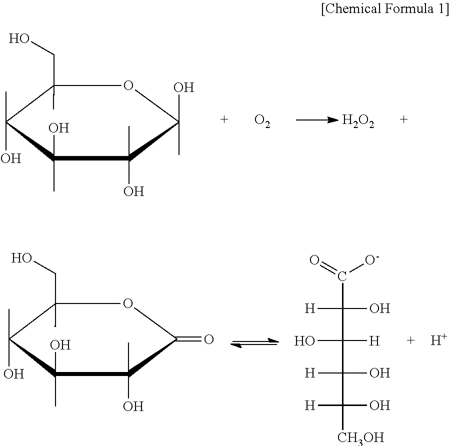

The insulating layer 550 absorbs hydrogen ions generated from the gluconate. As the insulating layer 550 absorbs hydrogen ions, a voltage value applied to the channel layer varies.

To this end, the insulating layer 550 may be formed of Al₂O₃ or Ta₂O₅ that sensitively sense H⁺. The insulating layer 550 may change a voltage applied to the channel layer according to a concentration of H⁺ to change a current amount flowing through the channel layer 540.

On the other hand, a surface treatment for fixing the enzyme may be carried out on the insulating layer 550.

According to an embodiment, as illustrated in FIG. 11A, a polymer compound for fixing the enzyme on the insulating layer may be used. Specifically, a mixture of the enzyme and the polymer compound may be coated on the insulating layer. Here, the polymer compound may be at least one of hydroxypropyl cellulose, methyl cellulose, hydroxyethyl cellulose, carboxymethyl cellulose, pullulan or chitosan. Furthermore, a ratio of the enzyme in the mixture may be 20-30 wt %.

According to an embodiment, as illustrated in FIG. 11B, an amine group, a carboxyl group, a silane group and the like for fixing the enzyme on the insulating layer may be treated. Specifically, a self-assemble monolayer (SAM) 560b may be coated on the insulating layer. For example, a cystamine solution capable of exposing an amine group may be applied on the insulating layer, and the cystamine solution, with its concentration ranging between 5-30 mM, uses distilled water or dimethyl sulfoxide as a solvent. Here, A glutaraldehyde solution may be used for a linker, and the glutaraldehyde solution uses distilled water as a solvent, and a concentration of which is 0.01-5 wt %.

Hereinafter, a sensor including the foregoing field-effect transistor will be described.

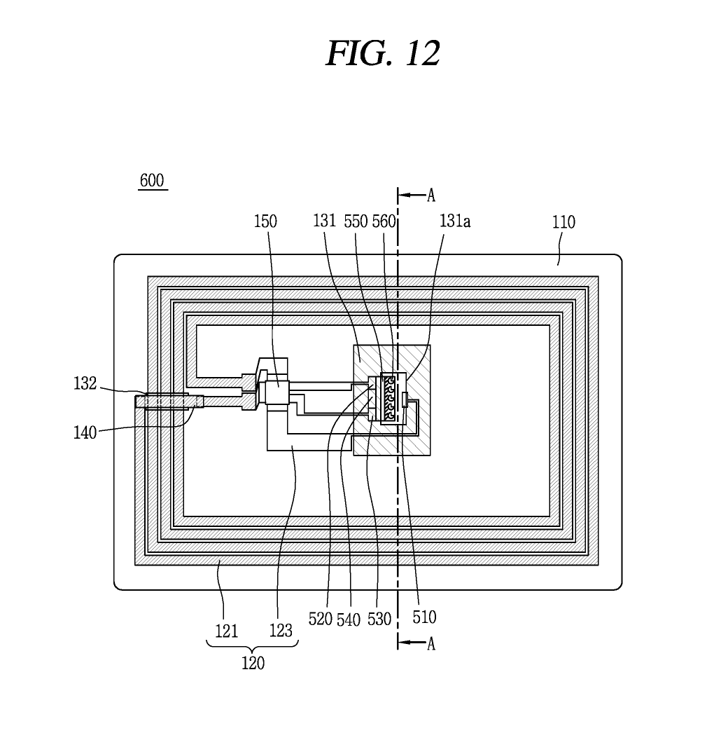

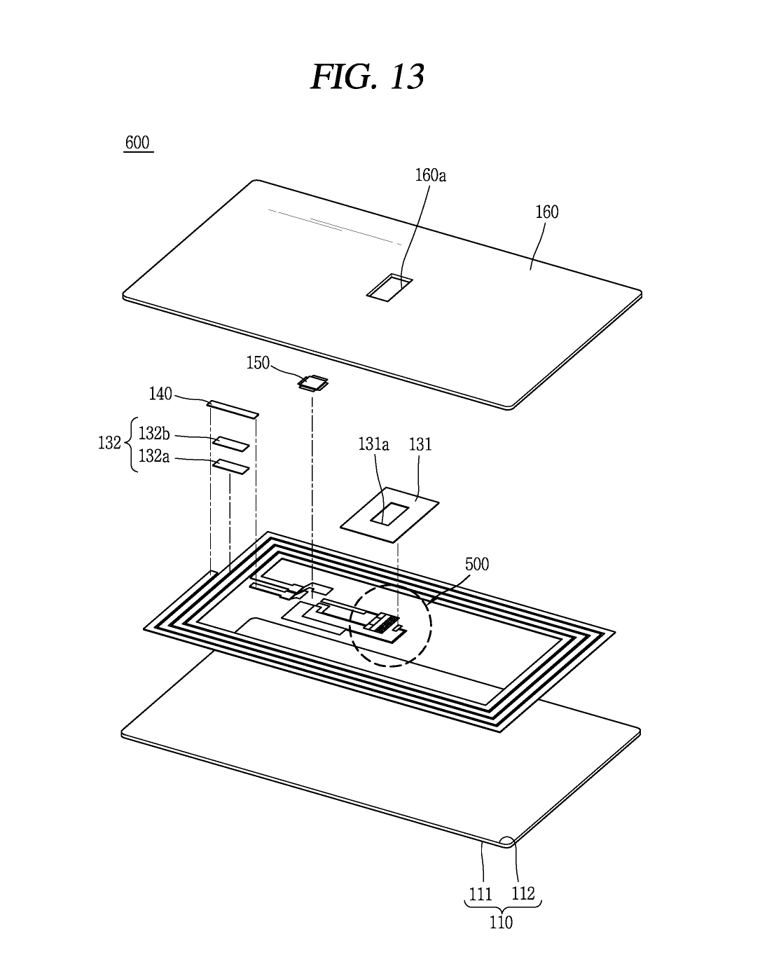

FIG. 12 is a circuit diagram illustrating a sensor including a field-effect transistor according to the present disclosure, and FIG. 13 is an exploded view illustrating the sensor of FIG. 12.

As illustrated in FIGS. 12 and 13, a sensor 600 has a form in which the sensing electrode 122 is replaced with a field-effect transistor in the sensor described in FIGS. 1 through 4. Accordingly, the description of the constituent elements of the sensor 600 which will be described below will be substituted by the description of FIGS. 1 through 4 unless otherwise specified.

The sensor 600 may include a field-effect transistor 500 (hereinafter, referred to as a transistor 500) according to the present disclosure, a substrate 110, a conductive layer 120, a circuit insulating layer 131, an antenna insulating layer 132, an antenna bridge 140 and a device 150.

When the sensing unit is the transistor 500, the conductive layer 120 may include the antenna pattern 121 and the circuit line 123. In other words, when the sensing unit is the transistor 500, the conductive layer 120 does not include the sensing electrode 122.

On the other hand, the circuit line 123 is electrically connected to the device 150 for controlling the transistor 500. Specifically, the circuit line 123 connects the gate 510, the source 520 and the drain 530 electrode, included in the transistor 500 to the device 150. However, the shape of the circuit line 123 may not be necessarily limited to the one illustrated in FIG. 12, and may be implemented in any shape capable of connecting the gate 510, the source 520 and the drain 530 electrode to the device 150.

A wireless signal received through the antenna pattern 121 is transmitted to the device 150 through the circuit line 123. Furthermore, a voltage inputted to the transistor 500 is inputted to the transistor 500 through the circuit line 123.

When a voltage is applied to the gate 510, the source 520 and the drain 530 electrode of the transistor 500, a current flows along the channel layer 540, and the intensity of a current varies according to the concentration of the contact target material in contact with the transistor 500.

The device 150 converts the intensity of the current flowing through the channel layer 540 into data to transmit it to an external device. A method of allowing a sensor according to the present disclosure to operate in connection with the external device will be described in detail.

In one embodiment, a user may select the type of a contact target material to be detected using an external device 800.

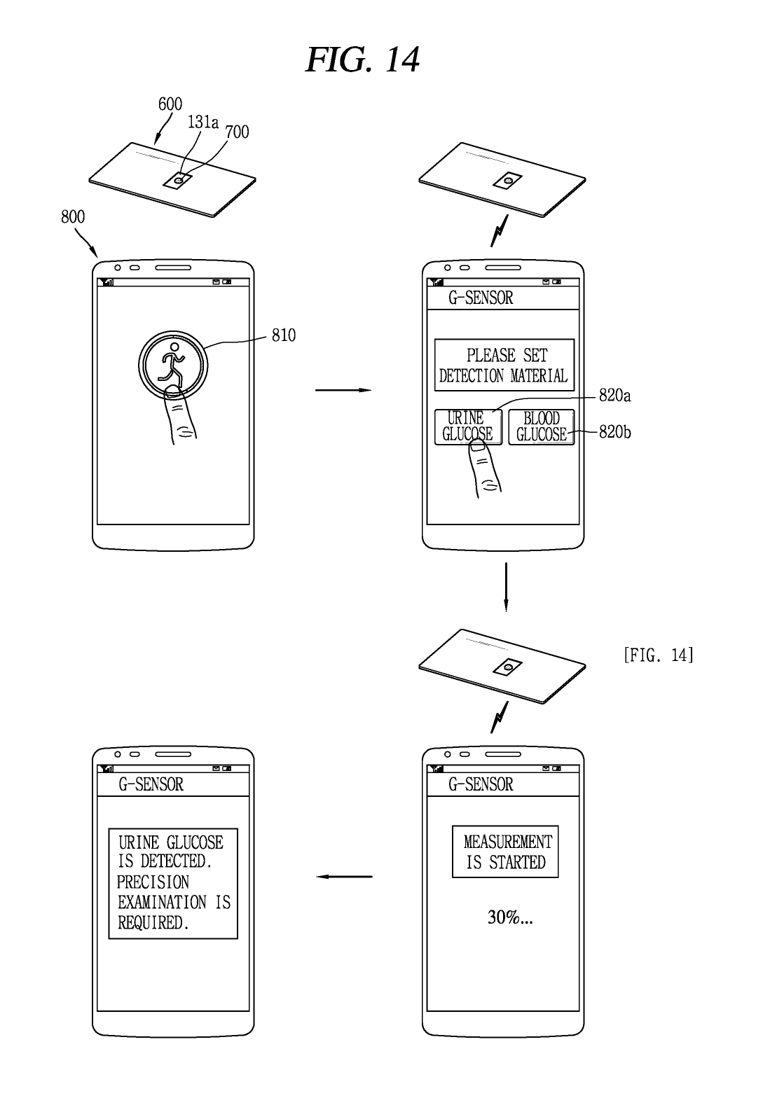

FIG. 14 is a conceptual view illustrating an embodiment of selecting a contact target material to be detected using the external device 800.

As illustrated in FIG. 14, the external device 800 may execute a preinstalled application based on a user input 810. When the application is executed and the sensor 600 approaches the external device 800 by less than a predetermined distance, the sensor 600 is driven. When the sensor 600 is driven, the external device 800 may transmit and receive data to and from the sensor 600.

On the other hand, the user may inject a foreign substance 700 into the window 131a. Here, the time point of injecting the foreign substance 700 may be prior to or subsequent to driving the sensor 600.

When the sensor 600 is driven, the sensor 600 transmits data to the external device 800 in proximity thereto. Here, data transmitted to the external device 800 may be data associated with the intensity of the current and the type of the sensor. The external device 800 may display a list 820a, 820b of detectable materials to the display unit.

The external device 800 may select a detection material based on a user input to the list, and determine whether the selected detection material exists or not using data associated with an intensity of a current received from the sensor 600 or calculate a concentration of the selected detection material. In other words, the external device 800 may process data associated with the intensity of a current received from the sensor 600 in a different way according to a user's input.

The configurations and methods according to the above-described embodiments is not limited to the foregoing sensor and a fabrication method thereof, and all or part of each embodiment may be selectively combined and configured to make various modifications thereto.

1. A sensor, comprising:
a substrate;
an antenna pattern formed to transmit and receive a wireless signal to and from an external device;
a sensing unit configured to be driven when the wireless signal is received through the antenna pattern and to generate a signal when in contact with a sensing target material; and
a circuit line electrically connected between the antenna pattern and the sensing unit,
wherein the antenna pattern and the circuit line are formed of a same material and on a same layer.

2. The sensor of claim 1, wherein the sensing unit is driven by generating direct-current power from the wireless signal received through the antenna pattern and transmit the signal to the external device.

3. The sensor of claim 1, wherein the circuit line comprises:
a power generation unit configured to generate direct-current power using the wireless signal received through the antenna pattern;
a controller configured to be driven by the direct-current power to input a voltage to the sensing unit;
a conversion unit configured to convert the signal generated from the sensing unit into a digital signal; and
a communication unit configured to transmit the digital signal to the external device through the antenna pattern.

4. The sensor of claim 1, wherein the sensing unit is an sensing electrode configured to cause an impedance change when in contact with the sensing target material, and the antenna pattern, the circuit line and the sensing electrode are formed on a surface of the substrate as a single conductive layer.

5. The sensor of claim 4, wherein the antenna pattern, the circuit line and the sensing electrode are formed of the same material.

6. The sensor of claim 4, wherein the sensor comprises a circuit insulating layer provided with a window to expose the sensing electrode, and disposed to cover at least part of the circuit line.

7. The sensor of claim 6, further comprising a first end portion and a second end portion in a lengthwise direction, and the circuit insulating layer is formed to cover the first end portion and the second end portion, and the window is formed to expose a region between the first end portion and the second end portion.

8. The sensor of claim 6, wherein a length of the sensing electrode is more than 400 μm, and
an exposure length of the sensing electrode exposed through the window is between 300 um and 2,000 μm.

9. The sensor of claim 6, wherein the circuit insulating layer has surface energy larger than that of the substrate.

10. The sensor of claim 4, wherein the single conductive layer comprises:
solid particles formed of at least one of silver (Ag), copper (Cu) or aluminium (Al) to have a spherical shape or a flake shape; and
at least one organic substance selected from a group consisting of polyethylene oxide (PEO) series, oleic acid series, acrylate series, acetate series and epoxy series.

11. The sensor of claim 4, wherein the single conductive layer has a pore.

12. The sensor of claim 4, wherein the substrate comprises a plastic layer having flexibility and a silica layer formed between the plastic layer and the conductive layer.

13. The sensor of claim 1, wherein the sensing unit is a field-effect transistor comprising a gate electrode, a source electrode, and a drain electrode, and wherein the field-effect transistor comprises:
a channel layer located between the source and the drain electrode;
an insulating layer located on the channel layer; and
a sensing layer located on the insulating layer,
wherein the sensing layer is separated from the gate electrode by a predetermined distance.

14. The sensor of claim 13, wherein the field-effect transistor is configured to cause a change in current value flowing along the channel layer when in contact with the sensing target material.

15. The sensor of claim 13, wherein the sensing layer is formed of a mixture of a predetermined enzyme and a predetermined high molecular weight compound.

16. The sensor of claim 15, wherein the predetermined enzyme comprises at least one of glucose oxidase or glucose dehydrogenase.

17. The sensor of claim 13, wherein the sensing layer comprises a self-assembled monolayer and a predetermined enzyme.

18. The sensor of claim 1, wherein the antenna pattern is extended in a two-dimensional spiral shape having a line width between 500 μm and 1,500 μm, and
a distance between lines that form the two-dimensional spiral shape is 300 to 700 μm.

19. A method of fabricating a sensor, the method comprising:
printing a conductive layer having an antenna pattern, a sensing electrode and a circuit line on one surface of a substrate with a single layer;
heat-drying the conductive layer;
printing, on a single layer, a circuit insulating layer that covers part of the circuit line and an antenna insulating layer that covers part of the antenna pattern;
curing the insulating layer;
printing an antenna bridge on the antenna insulating layer;
heat-drying the antenna bridge; and
bonding a device electrically connected to the circuit line to the substrate.

20. The method of claim 19, wherein the conductive layer is formed by printing ink powder or paste, and wherein the composition of the ink powder or paste comprises:
40-70 weight percent of solid particles formed of at least one of silver (Ag), copper (Cu) or aluminium (Al) to have a spherical shape or a flake shape; and
30-60 weight percent of at least one organic substance containing a solvent and selected from a group consisting of polyethylene oxide (PEO) series, oleic acid series, acrylate series, acetate series and epoxy series,
wherein the solvent comprises at least one selected from a group consisting of acetone, allyl alcohol, acetic acid, acetol, methyl alcohol and benzene.
